(19) United States
(12) Patent Application Publication
(10) Pub. No.: US 2007/0231885 A1
(43) Pub. Date: Oct. 4, 2007

(54) Biomechanical device for producing a biomass

(75) Inventors: Chris E. Choate, Discovery Bay, CA (US); James H. Lord, Los Gatos, CA (US)

Correspondence Address: CARR & FERRELL LLP, 2200 GENG ROAD, PALO ALTO, CA, 94303, US

(73) Assignee: Norcal Waste Systems, Inc.

(21) Appl. No.: 11/584,680

(22) Filed: Oct. 19, 2006

Related U.S. Application Data
(60) Provisional application No. 60/749,352, filed on Dec. 9, 2005.

(51) Int. Cl. C12M 1/10

(52) U.S. Cl. 435/290.3

(57) ABSTRACT

A rotating drum is provided for the biological/mechanical conversion of organic waste material to a valuable biomass. An aerotolerant anaerobic bacteria within the rotating drum promotes the fermentation of the waste material, while the tumbling of the waste material due to the rotation of the drum mechanically shears the waste material. Air carrying volatile organic compounds released by the fermentation process can be collected from the drum and scrubbed to recover the volatile organic compounds. The cleansed air can then be recirculated back into the drum. The waste material can be screened prior to fermentation in the drum to remove a fraction of the smallest particles. The biomass produced by the drum can also be screened.

### CROSS-REFERENCE TO RELATED APPLICATIONS

This application claims the benefit of U.S. Provisional Patent Application No. 60/749,352 filed on Dec. 9, 2005 and entitled “Biomass Production from Waste Material for Energy Generation” which is incorporated herein by reference. This application is related to U.S. application Ser. No. 11/492,258 filed on Jul. 24, 2006 and entitled “Systems and Processes for Treatment of Organic Waste Materials with a Biomixer,” which is also incorporated herein by reference.

### BACKGROUND OF THE INVENTION

1. Field of the Invention

The present invention relates generally to processing of waste materials, and more particularly to systems and processes for handling organic waste materials.

2. Description of the Prior Art

Landfilling has traditionally been the method of waste handling, but landfilling can cause environmentally unacceptable pollution discharges to the water and, as real estate values increase, is considered to be an undesirable use of land. Thus, current waste management strategies seek to limit the amount of refuse directed to landfills. Recycling and composting programs have become widely accepted for both commercial and residential waste to reduce the demands on landfills.

Generally, recycling programs require separating the waste by type, either at a point of collection (source separated) or further along, such as at a transfer station. Recyclable components can include glass, metals, and plastics, while compostable components can include, for example, agricultural wastes, plant matter, food stuffs, wood, cardboard, and paper. Once separated, waste materials are commonly referred to as “source separated,” and source separated materials that are collected together from separate collection points constitute a “single stream.”

Compost facilities have been built to process non-recyclable waste, either in the form of municipal solid waste with provisions for contamination removal, or source separated organic waste. An alternative to composting for non-recyclable waste streams are refuse-to-energy plants where material is burned to create energy. Refuse-to-energy plants first process waste by grinding and then burning the ground material. Although efforts are made to separate out hazardous materials from the waste stream, these plants have had a history of emissions and operational problems related to contaminants. The residual ash created from this burning has also, in some cases, been found to be hazardous.

Anaerobic digestion presents another alternative for handling organic waste materials. The primary objective of anaerobic digestion is the production of a mixture of hydrocarbon gases (“biogas”), which may be utilized as an energy source to generate electricity and/or heat. Any solid material remaining at the completion of the anaerobic digestion process is typically disposed of by conventional landfilling or composted into a soil amendment.

Because of the high capital costs associated with anaerobic digestion equipment, and the environmental issues associated with refuse-to-energy plants, composting has become the dominant method in the United States for the management and re-use of organic waste materials generated in rural and suburban settings. The growing use of composting as a preferred alternative to disposal of organic waste material has also created some environmental problems. These problems include emissions of noxious gases and ozone pre-cursors, runoff from the compost facility, and high energy consumption during material processing. These problems may become particularly acute if the organic waste material contains large amounts of food waste or other high moisture content waste.

Commercial-scale composting is also subject to a variety of financial considerations including capital investment related to accommodating peak seasonal feedstock deliveries, compost process time, and controlling the timing of compost production to match the seasonal demand of the agricultural industry and other compost buyers. Further, the compost produced by these facilities is a low-value product, therefore municipalities have to pay to have the waste accepted.

### SUMMARY

An exemplary system for processing organic waste material to a biomass product comprises a rotatable drum and an air system. The drum is sloped relative to the horizontal, i.e., level ground, and includes a feed end and a discharge end and breaker bars extending inwardly from an interior surface of the drum. The drum also includes an aerotolerant anaerobic bacteria to facilitate fermentation of the organic waste material. The air system is configured to blow air into the discharge end of the drum. In some embodiments, the air system can also include an air scrubber configured to remove volatile organic compounds, such as volatile fatty acids, from air discharged from the feed end of the drum. In some of these embodiments, the air system further includes a blower configured to recirculate air from the air scrubber to the discharge end of the drum. Loading equipment provided to load the waste material into the drum can, in some embodiments, also serve to collect the air from the feed end of the drum and therefore comprise part of the air system.

An exemplary method for processing organic waste material to a biomass product comprises fermenting a biodegradable fraction of the waste material with an aerotolerant anaerobic bacteria in a rotating drum, and controlling the environment within the rotating drum. Controlling the environment includes maintaining the oxygen content of the air within the drum below the ambient oxygen concentration, maintaining an acidic pH of the waste material within the drum proximate to a discharge end thereof, and maintaining the moisture content of the waste material within the drum in the range of 40% to 60%. Maintaining the oxygen content of the air within the drum can include, in some embodiments, removing air from a feed end of the drum and recirculating the air to the discharge end of the drum. Controlling the environment within the rotating drum can further include maintaining the temperature within the drum from about 130° F. at a feed end of the drum to about 165° F. at the discharge end. In some embodiments, the method further comprises adjusting a carbon to nitrogen ratio of the waste material to within a range of about 20:1 to about 40:1 prior to fermenting the biodegradable fraction of the waste material.

### BRIEF DESCRIPTION OF THE FIGURES

FIG. 1 is a schematic representation of an exemplary biomixer according to an embodiment of the invention.

FIG. 2 is a perspective view of a discharge end of the biomixer of FIG. 1.

FIG. 3 is partial cross-section of the biomixer of FIG. 1.

FIG. 4 is a perspective view of a feed end of the biomixer of FIG. 1.

FIG. 5 is a schematic representation of an exemplary biomass production facility including a biomixer according to an embodiment of the invention.

### DETAILED DESCRIPTION

Apparatus and methods for the mechanical/biological treatment of organic waste materials are provided herein. These apparatus, termed “biomixers,” employ a combination of mechanical shearing and biological activity in a controlled environment to produce biomasses suitable for anaerobic digestion and other purposes. An embodiment of the invention is directed to a rotating drum that includes bacteria capable of facilitating a fermentation process. Additionally, the embodiment includes an air system to move air through the rotating drum. Adjustments to the air flow through the drum can be used to control the fermentation process therein. The air system can also be used to recover volatile fatty acids from the environment of the drum.

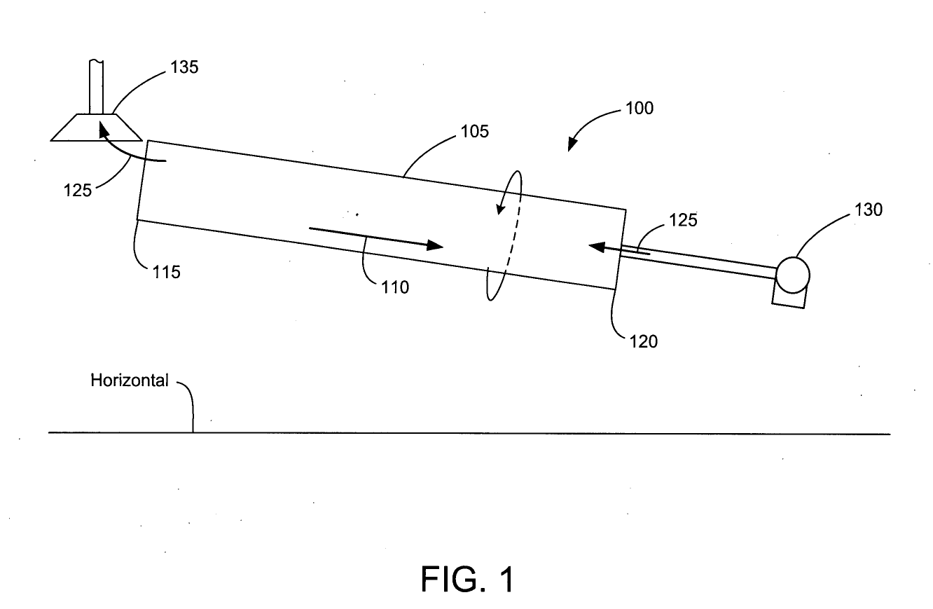

An exemplary biomixer 100 for producing a biomass is described with reference to FIG. 1. The biomixer 100 comprises a rotatable drum 105 that in operation is sloped relative to the horizontal so that waste material (represented by arrow 110) introduced at a feed end 115 traverses the biomixer 100 to a discharge end 120. FIG. 1 also shows an air system for moving air (represented by arrows 125) through the biomixer 100 and, in some embodiments, for recirculating and/or recovering volatile fatty acids from the air 125. Components of the air system that are shown in FIG. 1 include an air injector 130, such as a blower, and an air collection device 135, such as a hood. The air system is discussed in more detail elsewhere herein.

A suitable drum 105 for the biomixer 100 comprises a cylinder approximately 8 feet to 16 feet in diameter with a length of up to about 15 times the diameter. One suitable material for the drum is mild steel. A drum 105 as described can be supported by two drum support brackets (not shown). Drums with larger ratios of length to diameter may require additional drum support brackets. A drum drive unit (not shown) is provided to rotate the drum in a range of about ¼ to 2 revolutions per minute.

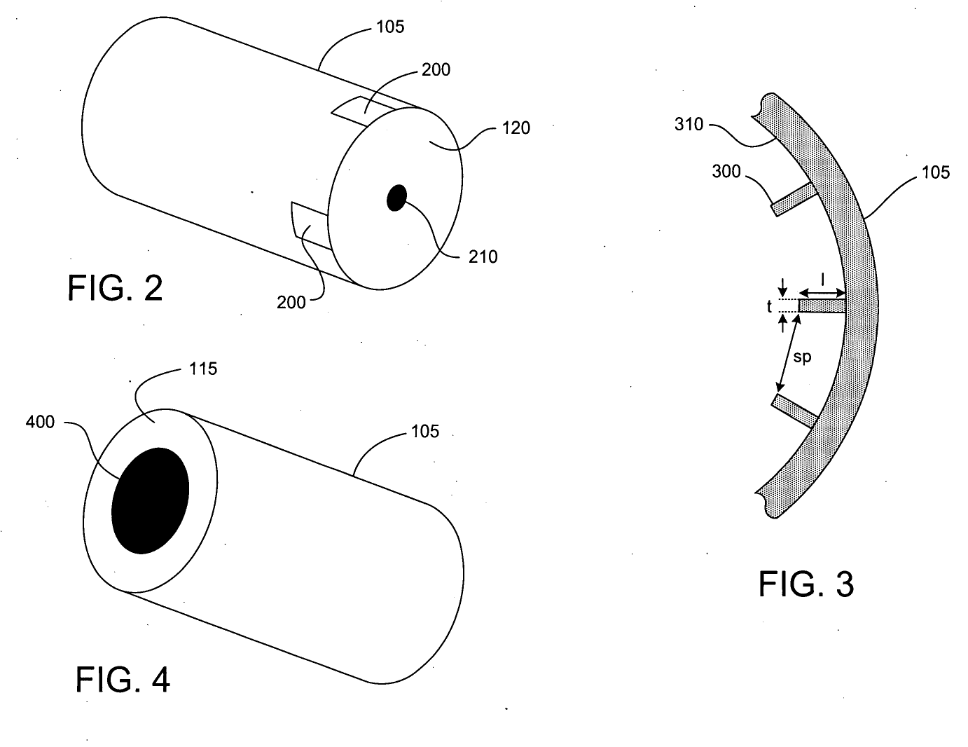

As shown, the drum 105 can be maintained at an angle relative to the horizontal with the discharge end 120 lower than the feed end 115. Under operating conditions, the slope of the drum 105 is about 3/16 of an inch per foot of length, in some embodiments, but can be increased or decreased to adjust the rate with which waste material traverses the drum 105. To protect against excessive corrosion of the 105 drum, cathodic protection, liners, and/or coatings can be applied to the inside of the drum. The drum 105 can also include access manholes, sampling ports, monitoring ports, and discharge ports, such as the discharge ports 200 in the perspective view of the discharge end 120 of the biomixer 100 shown in FIG. 2. Some embodiments employ four air actuated discharge ports 200, sized approximately 30 inches by 36 inches.

As also shown in FIG. 2, an air inlet 210 located near the center of the discharge end 120 of the drum 105 communicates with the air injector 130 (FIG. 1). Air 125 exiting the drum 105 from the feed end 115 (FIG. 1) can be recovered and scrubbed of volatile fatty acids and optionally returned to the drum 105 by the air injector 130. As discussed below, this recirculated air can have an advantageously lower oxygen concentration compared to the atmospheric oxygen level.

FIG. 3 shows a partial cross-section of the biomixer 100. As can be seen, some embodiments include breaker bars 300 that extend inwardly from an interior surface 310 of the drum 105. The breaker bars 300 are provided to agitate the waste material and provide additional shearing. Further, the breaker bars 300 can serve to protect the drum 105. Specifically, material that packs between breaker bars 300 helps to control corrosion and abrasion of the drum 105. In some embodiments the breaker bars 300 extend about 2 inches in length, 1, as measured from the interior surface 310 and have a thickness, t, of about ½ an inch. In some embodiments the breaker bars 300 are spaced, sp, about 4 inches to about 6 inches apart. In some cases lining of the drum 105 with a plastic material, stainless steel, or a special coating will allow the breaker bars 300 to be spaced as much as 4 to 6 feet apart. It will be appreciated that the breaker bars 300 can extend substantially the length of the drum 105 (ice., perpendicular to the plane of the drawing in FIG. 3). Further embodiments of the biomixer 100 also include spikes (not shown) extending inwardly from the interior surface 310 near the feed end 115. The spikes are useful to break open any bags in the waste material.

As shown in FIG. 4, an opening 400 is provided at the feed end 115 of the drum 105 for introducing waste materials into the drum 105. The opening 400 can be aligned with suitable loading equipment (not shown) that minimizes spillage of waste material. Air 125 is also discharged from the feed end 115 of the drum 105 through the opening 400. In some embodiments, the loading equipment seals against the opening 400 and includes separate discharge ports for discharging the air 125. Thus, in some of these embodiments, the loading equipment also serves as at least part of the air collection device 135 (FIG. 1).

In some embodiments, the drum 105 is loaded to about half full at the feed end 115, leaving a few feet of headroom at the discharge end 120. When loaded in this way, approximately two thirds of the volume of the drum 105 is filled by the waste material, leaving the remaining third empty to allow the waste material to tumble as the drum 105 rotates. A suitable retention time is about 1.5 days, but can range from about one to about three days. A suitable rotation rate is about half of a revolution per minute, though the revolution rate can be increased during loading and/or unloading to approximately one revolution per minute to accelerate the unloading process and to help move the waste material down the length of the drum 105 to facilitate loading.

As noted above, the rotating drum 105 includes bacteria capable of facilitating a fermentation process. In order to introduce the bacteria into the drum 105, the biological content of the waste material can be adjusted, for instance, by addition of select bacteria prior to being loaded into the biomixer 100. The added bacteria can either be a cultured bacteria, or can be a bacteria that is recovered from a biomass previously produced by the biomixer 100. In the latter case, a small fraction of the biomass produced by the biomixer 100 is recirculated back into the waste material being introduced into the biomixer 100. In some embodiments the small fraction of biomass added to the waste material is ten percent or less of the mass of the incoming waste material.

The added bacteria can include any bacteria capable of facilitating a fermentation process, such as aerotolerant anaerobic bacteria. Aerotolerant anaerobic bacteria are specialized anaerobic bacteria characterized by a fermentative-type of metabolism. These bacteria live by fermentation alone, regardless of the presence of oxygen in their environment. Exemplary aerotolerant anaerobic bacteria include species in the genera Desulfomonas, Butyrivibrio, Eubacterium, Lactobacillus, Clostridium, and Ruminococcus.

In operation, a biodegradable fraction of the waste material, typically consisting primarily of paper and other organic components, is converted in the biomixer 100 to a partially hydrolyzed biomass by mechanical breakdown and fermentation. The paper fraction of the waste material becomes wet and is broken into increasingly smaller pieces by the mechanical action. Other organic components are likewise sheared by the tumbling action within the slowly rotating biomixer 100. At the same time, aerotolerant anaerobic bacteria in the low oxygen environment within the biomixer 100 facilitate fermentation of the biodegradable fraction. The fermentation results in the partial hydrolysis of the biodegradable fraction into volatile fatty acids and their precursors.

As noted above, the environment in the biomixer 100 is controlled to facilitate the fermentation process caused by the aerotolerant anaerobic bacteria. The environment is primarily determined by the composition of the waste material, including the choice of aerotolerant anaerobic bacteria, the rate of air flow through the drum 105, and the oxygen concentration of the air. In some embodiments the oxygen concentration of the discharged air (as it leaves the feed end 115) is below 3.0% and can be as low as about 0.5%. Within the biomixer 100 an oxygen level gradient can vary from about 0.5% near the feed end 115 to about 5.0% at the discharge end 120. Recirculating the discharged air back into the biomixer 100 helps maintain the low oxygen concentration within the biomixer 100.

As the waste material traverses the biomixer 100 towards the discharge end 120, both the production of volatile fatty acids from the waste material increases, and the pH of the waste material drops to about 5.5 or lower. A pH range from the feed end 115 to the discharge end 120 can vary from about 8 to about 4.5. If necessary, the pH of the waste material can be made more basic prior to being introduced to the drum 105 in order to raise the endpoint pH at the discharge end 120. While this can serve to protect the biomixer 100 from corrosion damage, raising the endpoint pH may also reduce the efficiency of the fermentation process.

Also, as the waste material traverses the biomixer 100, and the fermentation process increases, the temperature of the waste material also increases. A suitable temperature for the fermentation process is about 145° F. but the temperature can range from about 130° F. at the feed end 115 to about 165° F. at the discharge end 120. While the moisture content at the feed end 115 can be about 60%, heating the waste material causes some of the moisture to evaporate and be removed from the biomixer 100 by the air flowing therethrough. However, even though some moisture is lost as the waste material traverses the biomixer 100, other mass is also lost, for example, through the volatilization of volatile fatty acids. The overall result is that the moisture content of the waste material will range from about 60% at the feed end 115 to as low as about 40% at the discharge end 120, though a typical final moisture content is around 50%.

It will be appreciated that the biomixer 100 can also include sensors to measure moisture, oxygen content, pH, and the temperature of the environment at different locations within the biomixer 100. Conditions within the biomixer 100 can also be determined from readings made at locations outside of the biomixer 100. For instance, sensors can measure the moisture, oxygen content, and temperature of the air entering and exiting the biomixer 100. Samples of the waste material can also be withdrawn for compositional analysis through sampling ports in the drum 105.

Based on readings from the sensors and/or other measured process information, various parameters can be controlled to keep the moisture level, oxygen content, pH, and temperature in the biomixer 100 within desired ranges. These parameters can include the rotation speed of the drum 105, the rates of loading and unloading, the slope of the drum 105 relative to the horizontal, the air pressure at the discharge end 120, the moisture and oxygen content of the air being introduced into the biomixer 100, the pH and moisture content of the material being loaded into the biomixer 100, and so forth. In particular, the rate at which air is blown into the drum 105 can be controlled, for example, by being cycled on a timer or by an electronic controller configured to control the air flow rate based upon sensor measurements.

For example, the composition of the waste material can be optionally adjusted as needed to obtain a more optimal mixture for processing within the biomixer 100. For instance, drier material such as paper can be added where the moisture content of the incoming waste material is too high. Alternately, wetter materials or water can be added to the incoming waste material to increase the moisture content. A suitable moisture content for the waste material being introduced into the biomixer 100 is about 60% but can vary between about 50% to about 65%.

Similarly, other materials can be added as needed to adjust factors such as the pH, the carbon to nitrogen ratio, and the biological content of the waste material. For instance, additional carbon or nitrogen can be added in the form of select waste or chemicals. A suitable carbon to nitrogen ratio is about 30:1, but can vary within a range of about 20:1 to about 40:1. A neutral or slightly acidic pH in the range of 5 to 6 is also preferred. The pH can be made more basic, for example, by isolating and removing low pH waste or by adding select high pH waste. Similar techniques can be employed to lower the pH. The pH can also be adjusted by adding commercially available acids or bases.

The rate of mixing, temperature, oxygen content, and retention time in the drum 105 can also be controlled. Some of these parameters can be controlled, for instance, by adjusting the rotational speed of the drum 105, the slope of the drum 100 relative to the horizontal, the air flow through the drum 105, and the rates of feeding into, and discharge from, the drum 105. Additionally, the temperature, moisture content, and oxygen content of the air that is introduced into the drum 105 can also be controlled.

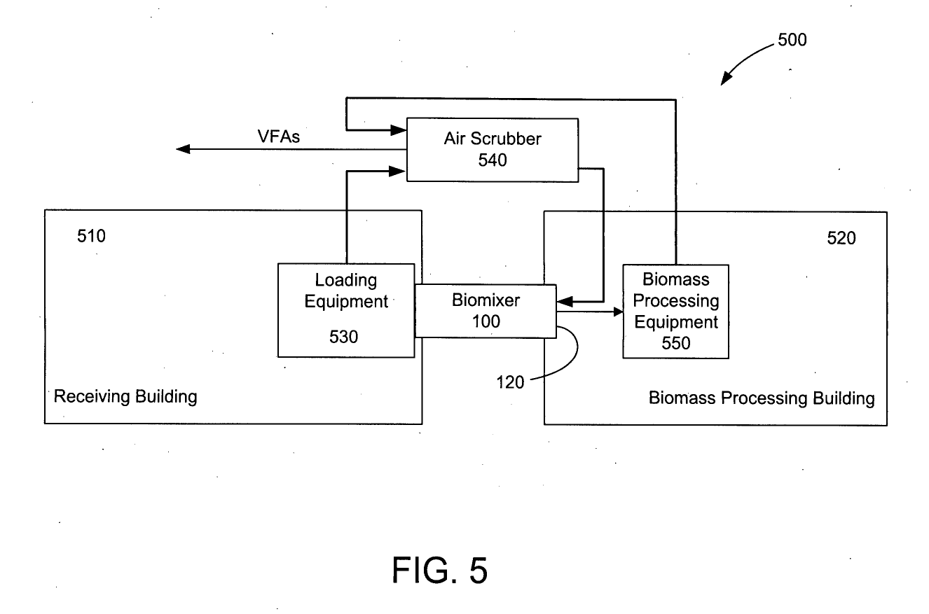

FIG. 5 is a schematic representation of an embodiment of a biomass production facility 500 including a biomixer 100. The biomass production facility 500 comprises a receiving building 510 and a biomass processing building 520. In this embodiment the biomixer 100 spans a distance between the buildings 510, 520 but, except for the feed and discharge ends 115, 120, is not itself housed within a building. Bladders, hoods, or other means, can be employed to seal around the biomixer 100 to keep odors from escaping from the buildings 510, 520. Each of the buildings 510, 520 can also include a negative pressure system that includes a biofilter to remove odors from the air. In the alternative, the entire facility can be housed in a single building rather than having the biomixer 100 span the distance between two buildings.

The receiving building 510 can include a tipping floor for receiving waste materials such as source separated organic waste or municipal solid waste. The receiving building 510 can also include sorting and screening areas and equipment. Sorting, which can be performed mechanically, by hand, or through a combination thereof, can be employed to remove hazardous materials such as batteries, problematic and/or unprocessable items such as construction materials, and recyclable materials. Screening, such as with a trommel, can be employed to classify the waste material according to size into waste streams of “overs” and “unders.”

In some embodiments, the overs are directed to the biomixer 100 while the unders are directed to other processing, such as anaerobic digestion. It will be appreciated that certain source separated waste may require little or no sorting or screening and may be loaded directly into the biomixer 100. In other instances, where the overall moisture content of the incoming waste material is above the desired moisture content range for processing in the biomixer 100, removing the unders from the incoming waste material is beneficial as the unders typically contain a disproportionate amount of the moisture of the waste material. Thus, removing the unders from the incoming waste material serves both to lower the moisture content of the waste material entering the biomixer 100 to a more acceptable range, and reduces the total amount of waste material that needs to be processed.

The receiving building 510 also includes loading equipment 530 to transfer the waste material into the biomixer 100. The loading equipment 530, in some embodiments, can include a mixer for adjusting characteristics of the waste material, such as moisture content and composition, prior to loading the waste material into the biomixer 100. The mixer can be used, for example, to blend amendments into the waste material to adjust the pH or to add bacteria. The loading equipment 530, in some embodiments, employs a gravity conveyor system and/or a mechanical ram to load the waste material into the biomixer 100. Loading waste material into the biomixer 100 can either be actuated manually or automatically based on a timed or volume-based cycle. As provided above, in some embodiments, the loading equipment 530 can also serve as at least part of the air collection device 135 (FIG. 1) of the air system. For instance, air can be discharged from the biomixer 100 through a feed ram opening or through dedicated discharge ports in the loading equipment 530.

The air system can also comprise an air scrubber 540 configured to receive the air recovered from the biomixer 100 through the air collection device 135, for example, through a hood or the loading equipment 530 as in FIG. 5. The air scrubber 540 can be located outside of both buildings 510, 520, as in FIG. 5, or located within either of the buildings 510, 520. The air scrubber 540 is configured to recover volatile organics from the air, thus suitable systems for the air scrubber 540 include water scrubbing systems. As the solubility of volatile fatty acids is higher in alkaline solutions, capture of volatile fatty acids can be improved by raising the pH of the water in the water scrubbing system.

Air collected from within the biomixer 100 can include a relatively high concentration of volatile organic compounds such as volatile fatty acids. The air scrubber 540 produces both cleansed air and concentrated volatile organic compounds in water. The volatile organic compound concentrate, preferably once saturated, can be directed to an energy recovery process or returned to the biomixer 100 through the loading equipment 530. The cleansed air from the air scrubber 540, having come initially from the low-oxygen environment within the biomixer 100, can have a lower oxygen content than the ambient concentration. In some embodiments, as shown in FIG. 5, the cleansed air is returned to the discharge end 120 of the biomixer 100 to help keep the oxygen concentration low within the biomixer 100. The oxygen content of the air entering the biomixer 100 can be further adjusted by adding ambient air to raise the oxygen level, or by adding an inert gas such as nitrogen to further dilute the oxygen level. Alternatively, rather than return the cleansed air to the biomixer 100, where a negative pressure system including a biofilter is employed to remove odors from the air within either of the buildings 510, 520, the cleansed air can also be directed into the biofilter and released to the atmosphere.

The biomass processing building 520, in some embodiments, includes biomass processing equipment 550 for screening and/or sorting the biomass produced by the biomixer 100. The biomass processing equipment 550 can include a trommel, for example, to classify the biomass into overs and unders for further processing. For instance, the overs can be directed to a composting facility while the unders are directed to an anaerobic digester. Suitable mesh sizes for the trommel range from about 1 to 3 inches.

While in some embodiments sorting and/or screening is performed on the waste material in the receiving building 510 prior to processing in the biomixer 100, in other embodiments sorting and/or screening is instead performed on the biomass product by the biomass processing equipment 550 in the biomass processing building 520. In still other embodiments sorting and/or screening is performed on both the waste material and the biomass product. As shown in FIG. 5, air from the biomass processing equipment 550 can also be directed to the air scrubber 540 as further processing of the biomass releases additional volatile fatty acids into the air.

In the foregoing specification, the present invention is described with reference to specific embodiments thereof, but those skilled in the art will recognize that the present invention is not limited thereto. Various features and aspects of the above-described present invention may be used individually or jointly. Further, the present invention can be utilized in any number of environments and applications beyond those described herein without departing from the broader spirit and scope of the specification. The specification and drawings are, accordingly, to be regarded as illustrative rather than restrictive. It will be recognized that the terms “comprising,” “including,” and “having,” as used herein, are specifically intended to be read as open-ended terms of art.

What is claimed is:

1. A system for processing organic waste material to a biomass product, the system comprising:
a rotatable drum, sloped relative to the horizontal, and including
a feed end and a discharge end,
breaker bars extending inwardly from an interior surface of the drum, and
an aerotolerant anaerobic bacteria; and
an air system configured to blow air into the discharge end of the drum.

2. The system of claim 1 wherein the drum further includes spikes extending inwardly from the interior surface of the drum proximate to the feed end.

3. The system of claim 1 wherein the aerotolerant anaerobic bacteria includes a species in a genera selected from the group consisting of Desulfomonas, Butyrivibrio, Eubacterium, Lactobacillus, Clostridium, and Ruminococcus.

4. The system of claim 1 wherein the air system includes an air scrubber configured to remove volatile organic compounds from air discharged from the feed end of the drum.

5. The system of claim 4 wherein the air system further includes a blower configured to recirculate air from the air scrubber to the discharge end of the drum.

6. The system of claim 4 wherein the air scrubber is a water scrubber having water with a basic pH.

7. The system of claim 1 further comprising loading equipment configured to load the waste material into the drum.

8. The system of claim 7 wherein the air system also comprises the loading equipment.

9. The system of claim 1 further comprising a receiving building and a biomass processing building, wherein the drum spans a distance between the buildings.

10. The system of claim 1 further comprising a trommel configured to receive the biomass product from the discharge end of the drum.

11. A method for processing organic waste material to a biomass product, the method comprising:
fermenting a biodegradable fraction of the waste material with an aerotolerant anaerobic bacteria in a rotating drum; and
controlling the environment within the rotating drum, including
maintaining the oxygen content of the air within the drum below the ambient oxygen concentration,
maintaining an acidic pH of the waste material within the drum proximate to a discharge end thereof, and
maintaining a moisture content of the waste material within the drum in the range of 40% to 60%.

12. The method of claim 11 wherein maintaining the oxygen content of the air within the drum below the ambient oxygen concentration includes removing air from a feed end of the drum and recirculating the air to the discharge end of the drum.

13. The method of claim 11 wherein controlling the environment within the rotating drum further includes maintaining the temperature within the drum from about 130° F. at a feed end of the drum to about 165° F. at the discharge end.

14. The method of claim 11 further comprising adjusting a carbon to nitrogen ratio of the waste material to within a range of about 20:1 to about 40:1 prior to fermenting the biodegradable fraction of the waste material.

15. The method of claim 11 further comprising collecting volatile organic compounds from the air within the drum.

16. The method of claim 11 wherein a retention time of the waste material within the drum during fermentation is in the range of about one day to about three days.

17. The method of claim 11 further comprising adding to the waste material, prior to fermenting the waste material in the drum, an amount of biomass previously removed from the discharge end.

18. The method of claim 11 further comprising screening the waste material to remove unders prior to fermenting the waste material in the drum.

19. The method of claim 11 further comprising screening the biomass removed from the discharge end.
